(19) United States
(12) Patent Application Publication
(10) Pub. No.: US 2024/0407948 A1
(43) Pub. Date: Dec. 12, 2024

(54) VACUUM-BASED DELIVERY TOOL TO FACILITATE PLACEMENT OF THIN-FILM OCULAR IMPLANT IN OPEN OR MINIMALLY INVASIVE SURGICAL SITES

(71) Applicant: Avisi Technologies, Inc., Redwood City, CA (US)

(72) Inventors: Jeffrey Lorne Emery, Emerald Hills, CA (US); Rui Jing Jiang, Lexington, MA (US); Georgia Griggs, Philadelphia, PA (US)

(21) Appl. No.: 18/737,451

(22) Filed: Jun. 7, 2024

Related U.S. Application Data
(60) Provisional application No. 63/507,278, filed on Jun. 9, 2023.

(51) Int. Cl. A61F 9/007; A61B 17/00; A61B 17/30; A61B 17/32; A61B 17/34; A61F 9/00; A61M 25/00

(52) U.S. Cl. CPC A61F 9/00781; A61F 9/0017; A61B 2017/00115; A61B 2017/00199; A61B 2017/306; A61B 17/320016; A61B 2017/346; A61B 17/3468; A61M 25/007; A61M 25/0082

(57) ABSTRACT

Described herein are devices configured to achieve an efficient grip and delivery of a thin-film implant into a surgical site that is open or minimally invasive. An example device includes a tip portion configured to secure and protect a thin-film implant device during handling and installation into a surgical site; a handle portion configured to control a vacuum source; and a shaft portion configured to control and deliver vacuum from the vacuum source to the tip portion.

### CROSS REFERENCE TO RELATED APPLICATION

The application claims priority to U.S. Provisional Patent Application No. 63/507,278, filed on Jun. 9, 2023, entitled “VACUUM-BASED DELIVERY TOOL TO FACILITATE PLACEMENT OF THIN-FILM OCULAR IMPLANT IN OPEN OR MINIMALLY INVASIVE SURGICAL SITES,” the content of which is incorporated by reference herein in its entirety.

### BACKGROUND

Thin-film implants may present unique challenges for maintaining a proper grip with conventional surgical tools in that these surgical tools may damage the thin-film implant, be unable for a user to grip the implant in the proper orientation, or not maintain the implant in a planar manner. Conventional surgical tools may not be able to support the implant without damage as it is delivered, particularly if the delivery is minimally invasive and requires pushing through small incisions or tunnels in the tissues of a patient. Further, conventional tools may not achieve final proper placement of the implant, such as being flat, without wrinkles, creases or folding at the surgical site, without significant subsequent manipulation by a user using a variety of other tools.

Accordingly, there is a need to provide a device, system and method configured to achieve an efficient grip and delivery of at least one thin-film implant into a surgical site that is open or minimally invasive (e.g., an ocular surgical site).

### SUMMARY

Described herein are devices, systems and methods configured to manipulate and deliver at least one thin-film implant into a surgical site that is open or minimally invasive (e.g., an ocular surgical site). In one preferred embodiment, a delivery tool or device may be configured to use vacuum to secure e.g., a thin-film and fragile implant onto an optimized delivery tool structure, so as to secure and protect the implant during handling and installation into a surgical site, such as an eye of a patient. Control (e.g., reduction and/or regulated release) of the vacuum may enable the delivery of the implant into the surgical site.

Among other features, the present disclosure provides a convenient, reliable, and secure system having a tool or delivery device that is uniquely matched to a thin-film or small implant to ensure its successful installation into an open or minimally invasive surgical site. The systems and methods of the present disclosure reduce the variability associated with user applied forces through traditional tools such as forceps as well as enabling low profile or even minimally invasive delivery options for an implant that can be furled into a smaller configuration and expanded into a larger one.

In accordance with aspects, the present disclosure relates to a device, comprising: a tip portion configured to secure and protect a thin-film implant device during handling and installation into a surgical site; a handle portion configured to control a vacuum source; and a shaft portion configured to connect the handle portion and the tip portion and control and deliver vacuum from the vacuum source to the tip portion.

In some embodiments, the vacuum source may be housed in the handle portion of the device. In other embodiments, the vacuum source may be external to the device. The tip portion, the shaft portion, and the handle portion may be located substantially in a same plane and form a straight configuration. In another embodiment, the tip portion, the shaft portion, and the handle portion are located in different planes with the shaft portion including multiple bends along its length to offset a first plane of the tip portion from a longitudinal axis of a second plane of the handle portion. Further, the vacuum source may include a power supply, wherein the power supply may include at least one of a syringe pump, a battery, or alternating current source. In yet another embodiment, the tip portion of the device may be configured to include a blade tip implemented on a distal end to prepare surgical installation of the thin-film implant device. According to one implementation, the blade tip may be deployable and connected to the handle portion via an actuation member that is secured to an actuation lever within the handle portion.

In certain embodiments, the device may further comprise at least one pressure transducer positioned in a flow path of vacuum for detecting a level of vacuum of the vacuum source, and a display configured to display the level of vacuum detected by the at least one pressure transducer. In one embodiment, the display may be implemented and installed at one of the tip portion, the shaft portion, or the handle portion. In another embodiment, the display may be configured to display the level of vacuum via one or more analog signals or digital signals. The display may include a light-emitting diode (LED) display. Moreover, the one or more analog signals or digital signals may comprise visual or audio signals configured to indicate the level of vacuum detected by the at least one pressure transducer.

According to additional embodiments, the tip portion of the device may include a plurality of vacuum slots. The tip portion may have dimensions and a shape configured to accommodate different sizes of the thin-film implant device. For example, edges of the tip portion at distal and proximal faces may be rounded, beveled or chamfered to facilitate a smooth and easy entry and exit to and from the surgical site. Further, the tip portion may be made of biocompatible materials. According to an embodiment, the tip portion of the device may include an array of perforations configured to regulate vacuum delivery while maintain a support of the thin-film implant device. In further embodiment, the tip portion may include a tip-to-implant face configured to be non-planar. The tip-to-implant face may also include a woven mesh or weave of polymeric fibers. For example, the tip-to-implant face may include a shallow nest in which the thin-film implant device resides while at least a portion of a perimeter of the tip portion is higher than a top most plane of the thin-film implant device.

According to additional embodiments, the tip portion of the device may be configurable between a furled state and a unfurled state, wherein the tip portion may be rolled with the thin-film implant device adhered to a top surface of the tip portion in the furled state, and the tip portion may be in the unfurled state for a placement of the thin-film implant device into a surgical site. In one implementation, the tip portion may include one or more wires extending within the shaft portion and connected with at least one component within the handle portion, each wire having a first end secured on a selected location on the top surface of the tip portion and a second end attached to the at least one component, which may be configured to control tension of the one or more wires. In an embodiment, the at least one component may include a lever or a button implemented on the handle portion. Further, the tip portion of the device may be made of elastic or articulating parts of materials to enable the furled and unfurled states of the tip portion. In a further embodiment, the tip portion may include one or more elastic struts to enable the unfurled state.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTIONS OF THE DRAWINGS

The present application will become more fully understood from the detailed description and the accompanying drawings, wherein:

FIG. 1 is a perspective view of a treatment device, according to one embodiment.

FIG. 2 is a close-up view of section A of the treatment device shown in FIG. 1.

FIG. 3 is a cross-sectional view of the treatment device shown along line III-III in FIG. 2.

FIG. 4 is a perspective view of a treatment device, according to another embodiment.

FIG. 5A is a portion of a cross-sectional view of section A of the device shown in FIG. 4 according to one embodiment.

FIG. 5B is a portion of a cross-sectional view of section A of the device shown in FIG. 4 according to one embodiment.

FIG. 5C is a portion of a cross-sectional view of section A of the device shown in FIG. 4 according to one embodiment.

FIG. 6 is a diagram of a treatment device implanted in an anterior chamber and between conjunctival tissue and scleral tissue of a patient's eye, according to an example embodiment of the present disclosure.

FIG. 7 is a diagram showing conventional forceps gripping a thin-film implant for placement in an eye of a patient.

FIG. 8 is a diagram of a delivery tool or device for a treatment application, according to an example embodiment of the present disclosure.

FIGS. 9(A) and 9(B) are diagrams of a tip portion of the delivery device without and with a thin-film implant in position, respectively, according to an example embodiment of the present disclosure.

FIG. 10(A) is a diagram of a deployable surgical blade incorporated into the delivery device for a treatment application, according to an example embodiment of the present disclosure.

FIG. 10(B) is an implementation of a prototype of FIG. 10(A), according to an example embodiment of the present disclosure.

FIG. 11 is a diagram of a tip portion of the delivery device including slots supported by internal braces, according to an example embodiment of the present disclosure.

FIG. 12 is a diagram of a tip portion of the delivery device in a furled state and an unfurled state, respectively, according to an example embodiment of the present disclosure.

FIG. 13 is a diagram of an internal view of a tip portion of the delivery device including pull wires, according to an example embodiment of the present disclosure.

FIG. 14 is a diagram of an alternative configuration of a tip portion of the delivery device, according to an example embodiment of the present disclosure.

FIGS. 15(A), 15(B), 15(C), 15(D), 15(E), 15(F), 15(G), 15(H), 15(I), 15(J), 15(K), 15(L), and 15(M) are screenshots showing how the delivery device is used to handle and deliver at least one thin-film implant into a surgical site that is open or minimally invasive (e.g., an ocular surgical site), according to an example embodiment of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

It should be appreciated that the device, system and method of the present disclosure may be utilized in any one or more medical or surgical procedures that involve fragile thin-film like implant such as, for example cardiac surgery, anastomosis procedures, non-surgical procedures, endoscopic procedures, non-invasive procedures, invasive procedures, port-access procedures, fluoroscopic procedures, beating heart surgery, vascular surgery, neurosurgery, electrophysiology procedures, diagnostic and therapeutic procedures, ablation procedures, ablation of arrhythmias, endovascular procedures, treatment of one or more organs and/or vessels, cardiograms, pharmacological therapies, drug delivery procedures, delivery of biological agents, gene therapies, cellular therapies, cancer therapies, radiation therapies, genetic, cellular, tissue and/or organ manipulation or transplantation procedures, coronary angioplasty procedures, placement or delivery of coated or uncoated stents, placement of cardiac reinforcement devices, placement of cardiac assistance devices, atherectomy procedures, atherosclerotic plaque manipulation and/or removal procedures, emergency procedures, cosmetic procedures, reconstructive surgical procedures, biopsy procedures, autopsy procedures, surgical training procedures, birthing procedures, congenital repair procedures, and medical procedures that require manipulation and delivery of one or more fragile thin-film like implant into a surgical site.

In one embodiment, as will be described fully below, the present disclosure relates to holding, placement and delivery of a thin-film based ocular implant such as for treatment of glaucoma. A glaucoma drainage implant is a small device (i.e., a thin-film device) placed in an eye of a patient to treat glaucoma. Most glaucoma patients have abnormally high intraocular pressure (IOP) due to the patient's inability to drain excessive aqueous humor from the anterior chamber of the eye through the trabecular meshwork. If not reduced with adequate treatment, high IOP will continuously damage the optic nerve as the disease progresses, leading to loss of vision or even total blindness. During a glaucoma implant surgery, a tiny drainage hole may be made in the sclera of the patient's eye (the white part of the eye). This opening allows fluid to drain out of the eye under the delicate membrane covering the eyeball known as the conjunctiva. Locally applied medications or injections may be used to keep the hole open and a thin-film glaucoma drainage device is positioned on the outside of the eye under the conjunctiva to drain excessive fluid out of the eye and into a place where the capillaries and lymphatic system of the patient reabsorb it back into the body, thereby lowering the intraocular pressure.

To reduce scaring and post-operative patient discomfort, it is desired to make as small as incision of the conjunctiva as possible, ideally less than 3 millimeters (“mm”). However, the treatment device, in many embodiments, has a width between 3 and 10 millimeters, preferably around 5 mm to provide to adequate drainage of aqueous humor from an anterior chamber of a patient's eye. This means that the treatment device is wider than a desired incision width.

To enable a minimally invasive insertion, the treatment device is folded or furled around an insertion device. After the insertion device passes through the conjunctiva incision, the insertion device is configured to unfold or unfurl the treatment device so that it rests flat or nearly flat within the sub-conjunctival pocket.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The description of illustrative embodiments according to principles of the present application is intended to be read in connection with the accompanying drawings, which are to be considered part of the entire written description. In the description of embodiments of the application disclosed herein, any reference to direction or orientation is merely intended for convenience of description and is not intended in any way to limit the scope of the present application. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the structure be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the application are illustrated by reference to the exemplified embodiments. Accordingly, the application expressly should not be limited to such exemplary embodiments illustrating some possible non-limiting combination of features that may exist alone or in other combinations of features; the scope of the application being defined by the claims appended hereto.

Unless otherwise specified, all percentages and amounts expressed herein and elsewhere in the specification should be understood to refer to percentages by weight. The amounts given are based on the weight of the material. According to the present application, the term “about” means+/−5% of the reference value. According to the present application, the term “substantially free” means less than about 0.1 wt. % based on the total of the referenced value.

A “subject” herein may be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

### Treatment Device Embodiment

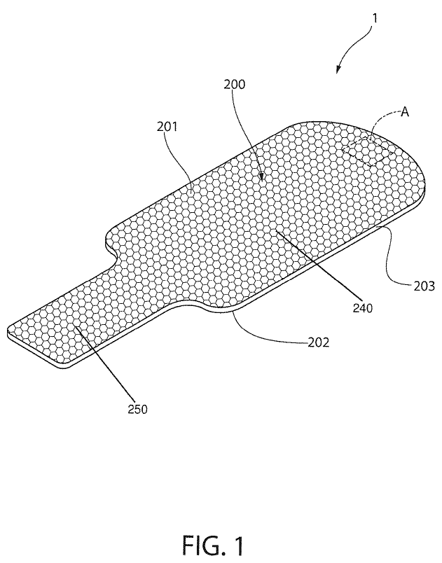

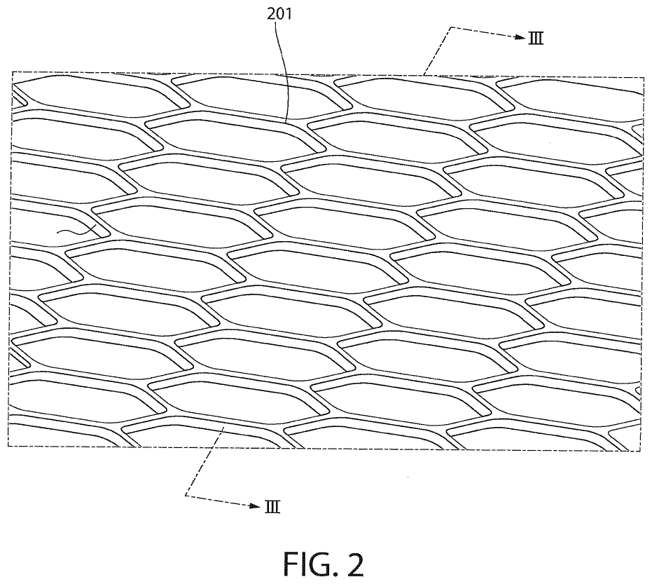

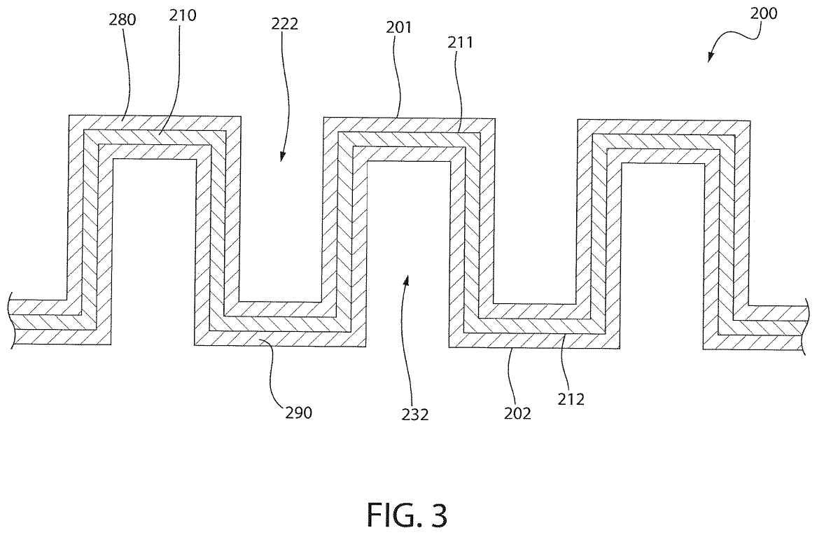

Referring to FIGS. 1-3, a treatment device 1 includes a plate structure 200, or simply plate, having a first major exposed surface 201 opposite a second major exposed surface 202 as well as side surface 203 extending there-between. The plate structure 200 can comprise an extension portion 250 and a main body portion 240.

The plate structure 200 may be formed of any material with appropriate characteristics for implantation and treatment. In some embodiments, the plate structure 200 can be formed of a metal, polymer, ceramic (e.g., aluminum oxide), other composite material, or a combination thereof. Metals can include, but are not limited to aluminum, titanium, zinc, platinum, tantalum, copper, nickel, rhodium, gold, silver, palladium, chromium, iron, indium, ruthenium, osmium, tin, iridium, or combinations, and alloys thereof. In some embodiments, alloys can include steel and nickel titanium such as Nitinol.

Polymers or polymer materials used to form plate structure 200 can include any of the polymers described herein.

Composites such as silicon composites can also be used. In one embodiment, a composite can include silicon nitride (Si₃N₄). The silicon nitride can have any known crystalline structure such as, but not limited to, trigonal α-Si₃N₄, hexagonal β-Si₃N₄, or cubic γ-Si₃N₄.

The plate structure 200, or plate, can have a thickness ranging from about 1 nm to about 1,000 nm, from about 1 nm to about 500 nm, from about 1 nm to about 400 nm, from about 100 nm to about 1,000 nm, from about 200 nm to about 1,000 nm, from about 300 nm to about 1,000 nm, from about 400 nm to about 1,000 nm, from about 1 nm to about 900 nm, from about 1 nm to about 800 nm, from about 1 nm to about 700 nm, from about 1 nm to about 600 nm, from about 300 nm to about 500 nm, from about 300 nm to about 600 nm, from about 400 nm to about 600 nm, from about 200 nm to about 600 nm, from about 200 nm to about 500 nm, or from about 50 nm to about 800 nm.

The plate structure 200 may comprise a multi-directional plate 210 comprising a first major surface 211 opposite a second major surface 212. The multi-directional plate 210 may form a plurality of topographical features (for example, a repeating honeycomb pattern) on each of the first major surface 211 and the second major surface 212. Each of the first and second topographies may independently comprise a plurality of channels 232 and/or a plurality of open-cells 222.

The plurality of channels 232 may be interconnected and can form a network of channels. The channels may be open or closed, allowing fluid to readily enter each channel of plurality of channels 232 and flow through it. The network may comprise intersecting channels in any suitable configuration to best help promote the flow of fluid across the plate structure 200 via the plurality of channels 232. In one embodiment, the channels 232 may be configured to form hexagonal patterns. Once treatment device 1, illustrated in FIG. 1, is implanted, fluid (e.g., aqueous humor) may be driven by a pressure gradient to flow through the channels and across the surface of plate structure 200.

In some embodiments, the channels 232 can include a ribbing pattern. The ribbing pattern and/or the geometry of the channels in the plate can be varied based on different severities of disease (e.g., mild, moderate, or severe glaucoma). In one embodiment, larger or smaller channels can be used to decrease intraocular pressure by different amounts. Changing intraocular pressure by a lower amount can decrease risk of hypotony (a condition that can exist if intraocular pressure is reduced too much) and increase efficacy at lowering pressure to a target level. In some embodiments, a device as described herein with smaller channels can decrease flow and decrease risk of hypotony. Likewise, larger channels can increase flow and allow the device to reduce intraocular pressure to a lower level.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210.

The first coating 280 may have a thickness ranging from about 0.1 μm to about 10 μm or about 0.1 μm to about 2 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

The plate structure 200 may further comprise a second coating 290 applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The second coating 290 may have a thickness ranging from about 0.1 μm to about 10 μm or about 0.1 μm to about 1 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

In some embodiments, the plate structure 200 may comprise only the first coating 280—i.e., no second coating. In other embodiments, the plate structure 200 may comprise only the second coating 290—i.e., no first coating. In other embodiments, the plate structure 200 may comprise the first coating 280 and the second coating 290, whereby the first and second coatings overlap to fully encapsulate the multi-directional plate 210. In such embodiments, the side surface 203 of the plate structure 200 may comprise at least one of the first coating 280 and the second coating 290.

In some embodiments, the first and second coating, and any edge coating, can be thicker than the plate itself. In some embodiments, the coating thickness can be one, two or three orders of magnitude thicker than the plate structure. However, in other embodiments, the plate can be thicker than each coating or the additive thickness of the two coatings.

Coatings described herein can be applied by any suitable deposition method, such as but not limited to, physical vapor deposition, chemical vapor deposition, atomic layer deposition, spray coating, spin coating, self-assembly, dip coating, or brushing.

The first coating 280 may be applied to the first major surface 211 by any suitable deposition method. In a non-limiting example, the first coating 280 may be applied to the first major surface 211 by chemical vapor deposition, physical vapor deposition, or plasma-enhanced chemical vapor deposition. In another non-limiting example, the first coating 280 may be applied to the first major surface 211 by atomic layer deposition. In another non-limiting example, the first coating 280 may be applied to the first major surface 211 by spray coating. In another non-limiting example, the first coating 280 may be applied to the first major surface 211 by dip coating. In another non-limiting example, the first coating 280 may be applied to the first major surface 211 by brushing.

The second coating 290 may be applied to the second major surface 212 by any suitable deposition method. In a non-limiting example, the second coating 290 may be applied to the second major surface 212 by chemical vapor deposition, physical vapor deposition, or plasma-enhanced chemical vapor deposition. In another non-limiting example, the second coating 290 may be applied to the second major surface 212 by atomic layer deposition. In another non-limiting example, the second coating 290 may be applied to the second major surface 212 by spray coating. In another non-limiting example, the second coating 290 may be applied to the second major surface 212 by dip coating. In another non-limiting example, the second coating 290 may be applied to the second major surface 212 by brushing.

The first coating 280 may be the same as the second coating 290. The first coating 280 and the second coating 290 may be different. The first coating 280 may be hydrophilic. The first coating 280 may be hydrophobic. The first coating 280 may be lipophilic. The first coating 280 may be lipophobic. The second coating 290 may be hydrophilic. The second coating 290 may be hydrophobic. The second coating 290 may be lipophilic. The second coating 290 may be lipophobic. Each of the first and second coatings 280, 290 may independently be continuous. Each of the first and second coatings 280, 290 may independently be discontinuous. In some embodiments, the first and second coatings 280, 290 may both be hydrophobic. In some embodiments, the first and second coatings 280, 290 may both be hydrophilic. In some embodiments, the first and second coatings 280, 290 may both be lipophilic or lipophobic.

The first coating 280 may be organic. The first coating 280 may be inorganic. The second coating 290 may be organic. The second coating 290 may be inorganic.

In some embodiments, the first coating 280 is hydrophilic and the second coating 290 is hydrophobic. In some embodiments, the first coating 280 is hydrophilic and the second coating 290 is hydrophilic. Having at least one of the first and/or second coating 280, 290 be hydrophobic may help prevent the treatment device 1 from inadvertently sticking to tissue during implantation.

In some embodiments, a purpose of a first and/or second coating is to increase the toughness of the device. Also, a first and/or second coating can increase biocompatibility of the device and/or decrease scarring by decreasing tissue and/or fibroblast adhesion. In some embodiments, the coatings described herein are hydrophobic and decrease tissue adhesion. In some embodiments, tissue adhesion can be reduced by greater than about 10%, greater than about 60%, greater than about 70%, greater than about 80%, greater than about 90%, greater than about 95%, greater than about 96%, greater than about 97%, greater than about 98%, or greater than about 99% when compared to an uncoated plate.

In a non-limiting embodiment, the first and/or second coating may comprise a polymer, such as a parylene polymer (poly(para-xylylene)) or a derivative thereof. In other embodiments, the first and/or second coating can include aluminum oxide, a biocompatible film, a porous coating, or a lubricious coating. In one embodiment, the parylene polymer is a chlorine modified poly(para-xylylene), or a fluorine modified poly(para-xylylene). In one embodiment, the parylene polymer can be parylene C, parylene D, parylene N, a derivative thereof or a combination thereof. In other embodiments, the first and/or second coating can include aluminum oxide.

In other embodiments, other polymer(s) can be used in addition to, in combination with, or instead of a parylene polymer and/or aluminum oxide. In some embodiments, other polymeric materials can include, but are not limited to rubber, synthetic rubber, silicone polymers, thermoplastics, thermosets, polyolefins, polyisobutylene, acrylic polymers, ethylene-co-vinylacetate, polybutylmethacrylate, vinyl halide polymers (for example, polyvinyl chloride), polyvinyl ethers (for example, polyvinyl methyl ether), polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides (for example, Nylon 66 and polycaprolactam), alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene (for example, Teflon), poly(ether-ether-ketone), poly lactides such as PLA, PLGA, PLLA, derivatives thereof, or combinations thereof.

The resulting treatment device 1 may comprise the first plurality of channels 222 present on the first exposed major surface 201 of the plate structure 200, wherein the first plurality of channels 222 are hydrophilic due to the presence of the first coating 280. The resulting treatment device 1 may comprise the second plurality of channels 232 present on the second exposed major surface 202 of the plate structure 200, wherein the second plurality of channels 232 are hydrophilic due to the presence of the second coating 290. As discussed, the hydrophilic channels may promote fluid flow through the channels after the treatment device 1 has been implanted into a subject's eye.

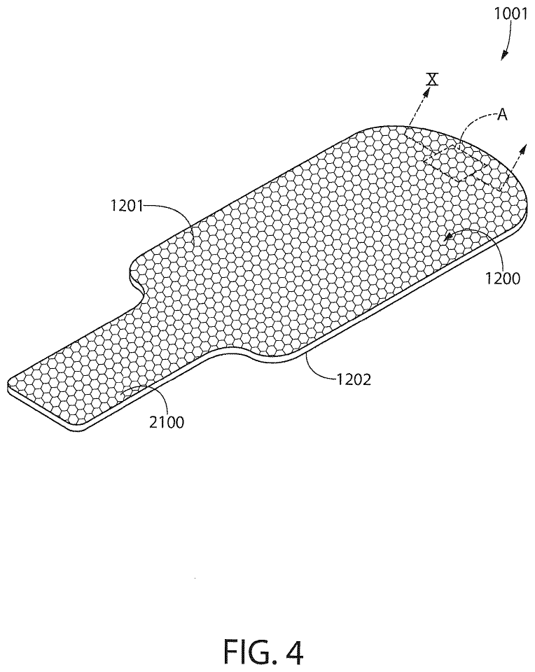

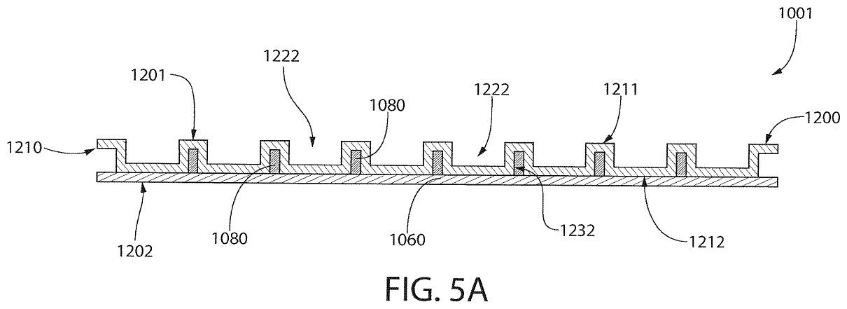

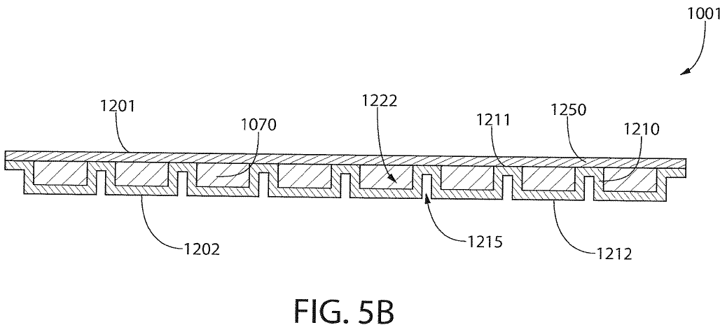

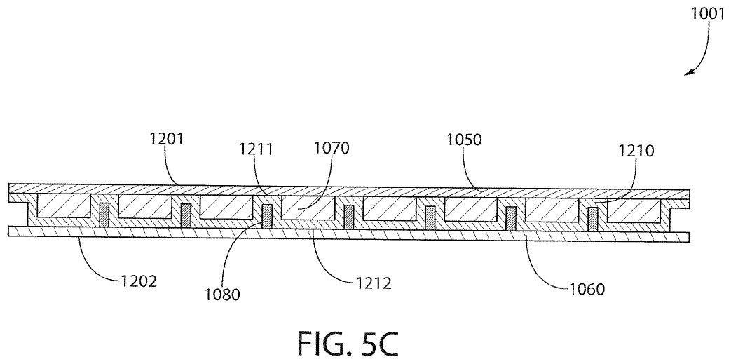

Referring to FIGS. 4, 5A, 5B, and 5C, generally, a treatment device 1001 is illustrated in accordance with another embodiment. The treatment device 1001 is similar to the treatment device 1 except as described herein below. The description of the treatment device 1 above generally applies to the treatment device 1001 described below except with regard to the differences specifically noted below. A similar numbering scheme will be used for the treatment device 1001 as with the treatment device 1 except that a “1000” series of numbering will be used.

The treatment device 1001 comprises a plate structure 1200 having a first exposed major surface 1201 that is opposite a second exposed major surface 1202. The plate structure 1200 may comprise a multi-directional plate 1210 comprising a first major surface 1211 opposite a second major surface 1212. The multi-directional plate 1210 may form a plurality of topographical features (for example, a repeating honeycomb pattern) on each of the first major surface 1211 and the second major surface 1212. Each of the first and second topographies may independently comprise a plurality of channels 1232 and/or a plurality of open-cells 1222.

Referring now to FIG. 5B, the plate structure 1200 may comprise a first drug-treatment delivery component 1070 present in the open voids created by the first topography formed by the first exposed surface 1211 of the multi-directional plate 1210. Specifically, the first delivery component 1070 may be present in the open voids created by the open-cells 1222 of first topography formed by the first major surface 1211 of the multi-directional plate 1210.

The first drug-treatment delivery component 1070 may comprise one or more active agents such as, but not limited to therapeutic and/or pharmacological components. The first drug-treatment delivery component 1070 may occupy some, all, or substantially all of the free volume present in the open-cells 1222 formed by the first topography.

In other embodiments, active agents can include any compound or drug having a therapeutic effect in a subject. Non limiting active agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, steroids, proteasome inhibitors, antibiotics, anti-inflammatories, anti-sense nucleotides, transforming nucleic acids, messenger ribonucleic acids, IOP lowering drugs, prostaglandins, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, cells, stem cells, liposomes, anti-metabolites such as mitomycin-C, combinations thereof, prodrugs thereof, pharmaceutical salts thereof, derivatives thereof, and the like.

The treatment device 1001 may further comprise a first coating 1050 applied to a first major surface 1211 of the multi-directional plate 1210. The first coating 1050 may cover both a first major surface 1211 of the multi-directional plate 1210 as well as a first drug-treatment delivery component 1070 that is present in the open-cells 1222 formed into the first major surface 1211 of the multi-directional plate 1210. The first coating 1050 may be in the form of a continuous film. The first coating 1050 may be flat. In other embodiments, the first coating 1050 may be conformal to the underlying pattern formed by the multi-directional plate 1210 and the first delivery component 1070.

Referring now to FIG. 5A, the plate structure 1200 may comprise a second drug-treatment delivery component 1080 present in the open voids created by the second topography formed by the second exposed surface 1212 of the multi-directional plate 1210. Specifically, the second delivery component 1080 may be present in the open voids created by the open-channels 1232 of the second topography formed by the second major surface 1212 of the multi-directional plate 1210. The second delivery component 1080 may be the same or different from the first delivery component 1070.

The second drug-treatment delivery component 1080 may comprise one or more therapeutic and/or pharmacological components-including but not limited to anti-inflammatory agents, steroids, antibiotics, analgesics. The second delivery component 1080 may occupy some, all, or substantially all of the free volume present in the channels 1232 formed by the first topography.

The treatment device 1001 may further comprise a second coating 1060 applied to a second major surface 1212 of the multi-directional plate 1210. The second coating 1060 may cover both the second major surface 1212 of the multi-directional plate 1210 as well as the second delivery component 1080 that is present in the open-channels 1232 formed into the second major surface 1212 of the multi-directional plate 1210. The second coating 1060 may be in the form of a continuous film. The second coating 1060 may be flat. In other embodiments, the second coating 1060 may be conformal to the underlying pattern formed by the multi-directional plate 1210 and the second delivery component 1080.

The second coating 1060 may be the same or different than the first coating 1050. For each of the first and the second coatings 1050, 1060, the resulting film may be formed from a slow-release material that dissolves slowly after exposure to aqueous humor or other biological fluids, thereby releasing the first delivery component 1070 from the channels 1232 of the treatment device 1001 after it has been implanted into a subject.

Referring now to FIG. 5C, in other embodiments, the treatment device 1001 may comprise both the first and the second drug-treatment delivery components 1070, 1080, as well as the first and the second coatings 1050, 1060 to encapsulate the first and second delivery components 1070, 1080.

In other embodiments, the plate structure 1200 may comprise at least one of the first coating 1050 and/or the second coating 1060 without the presence of the first and/or second delivery components 1070, 1080. In such embodiments, the first coating 1050 and/or the second coating 1060 may form a film that covers the open cells 1222 and/or the open channels 1232 created by the multi-directional plate.

The presence of the films resulting from the first and/or the second coating 1050, 1060 may enhance the overall strength of the resulting treatment device. Specifically, layered structure(s) of the films formed by the first and second coatings 1050, 1060, which are bonded to the first and second major surfaces 1211, 1212 of the multi-directional plate 1210, provide added mechanical integrity to the resulting treatment device.

Beyond achieving the baseline flexibility to conform to curvature of the eye, the addition of the first and/or second coatings 1050, 1060 may provide a mechanism that allows the overall treatment device to match the elastic modulus of surrounding tissues (e.g., conjunctival and scleral tissues) to maximize biocompatibility or biointegration. Findings in brain implant research confirm that the flexibility of implants in soft tissue improves compliance of the implant with microscale movements of surrounding tissue and reduces tissue displacement and trauma as well as facilitates implantation of the treatment device.

### Treatment Device Insertion Embodiment

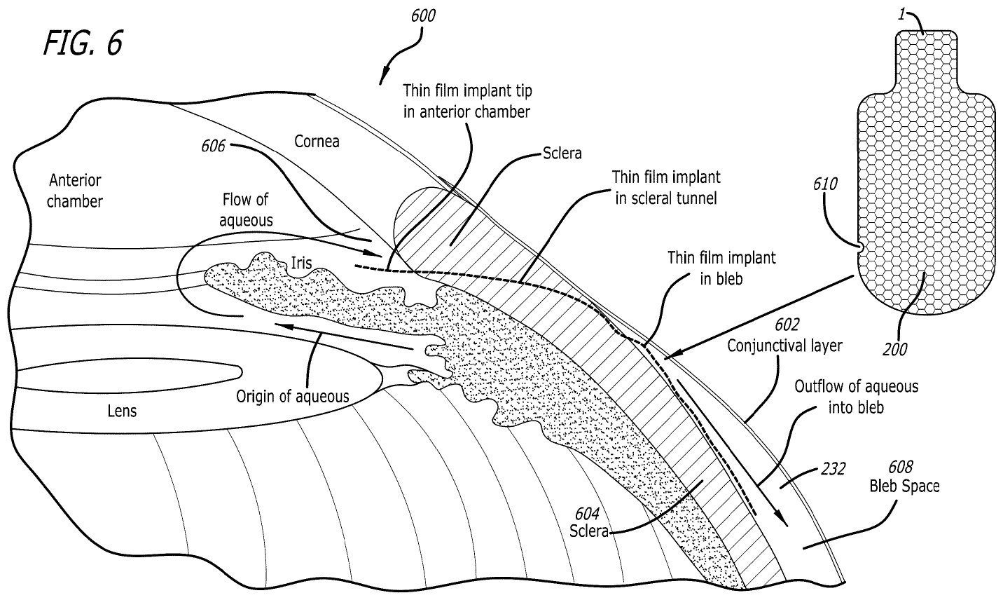

FIG. 6 is a diagram of the treatment device 1 implanted between conjunctival tissue 602 and scleral tissue 604 of a patient's eye 600, according to an example embodiment of the present disclosure. The treatment device 1 is a biocompatible ocular implant that includes a thin, flexible plate to facilitate safe, comfortable, and effective treatment. The treatment device 1 includes a plate structure 200 having a plurality of channels 232. The example channels 232 are configured to facilitate the draining of accumulated aqueous in the anterior chamber 606 of the eye 600 to a pocket (bleb) 608 that is located between the conjunctival tissue 602 and scleral tissue 604. This enables intraocular pressure from the accumulation of the aqueous in the anterior chamber 606 to be reduced. The removed aqueous in the pocket 608 is gradually reabsorbed by surrounding tissue, which enables further accumulating aqueous to be removed from the anterior chamber 606. This continuous draining of aqueous (e.g., glaucoma drainage) lowers pressure within the eye 600 and protects the optic nerve. The redundant channels 232 of the plate structure 200 prevent single-end clogging by scar tissue. Further, the thin profile of the plate structure 200 hinders tissue erosion.

FIG. 6 also shows the plate structure 200 including a notch 610 along a perimeter. While the notch 610 is shown on a lower left section of the plate structure 200, it should be appreciated that the notch 610 may be located at any location of the perimeter. Further, while one notch 610 is shown, the plate structure 200 may include two or more notches. The notch 610 is configured to facilitate proper installation and placement of the plate structure 200 within a patient's eye. The notch 610 may be indicative as to whether the channels 232 of the plate structure 200 are aligned upwards or downwards. The notch 610 accordingly provides confirmation to a clinician that the plate structure 200 is properly orientated.

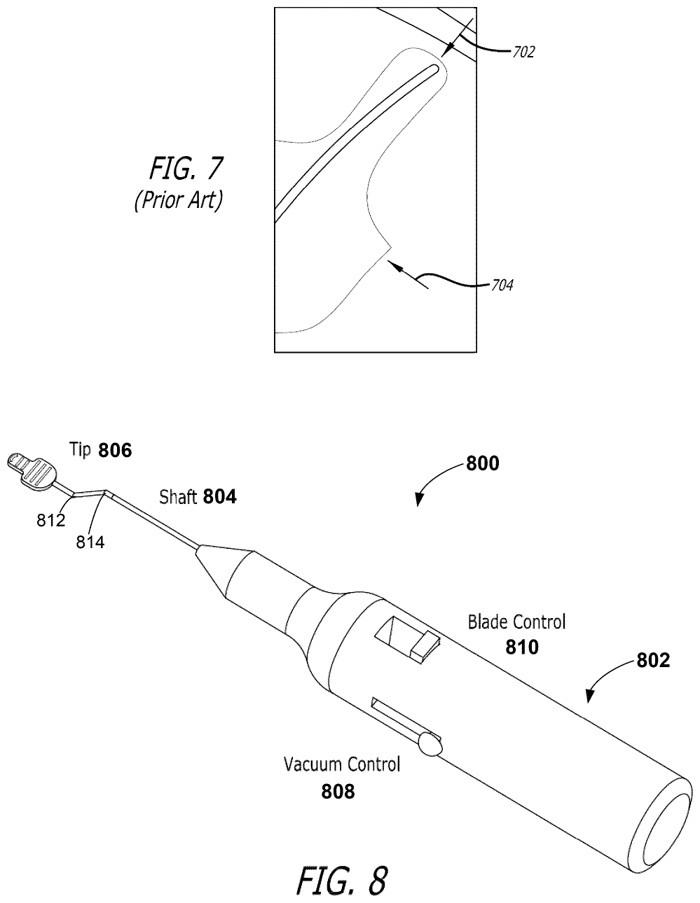

Conventional ocular surgical tools may include a variety of forceps, tweezers and spatulas. There is a broad spectrum of shapes and sizes of surgical tools, but none are well-suited to the delivery of a thin-film implant (e.g., treatment device 1 of FIG. 6) for treatment of glaucoma. The vast of majority of these tools have narrow tips and shafts, and rely on a user to maintain a constant, ideal grip on the implant without dropping or damaging the implant during the surgical procedure. Further, the contact area of conventional tools create a risk of stress concentration and/or damage by crushing or piercing, while at the same time leaving other critical areas of the implant unsupported. This lack of uniform, broad support across the entire surface of the thin-film implant may put the implant at risk of damage or mal-positioning if the surgical access site or final delivery location is tight-fitting or, in the case of minimally invasive delivery, the target location is even smaller than the implant itself. For example, FIG. 7 illustrates conventional forceps (arrow 702) gripping a thin-film implant (arrow 704) for placement in the eye of patient during a glaucoma implant surgery.

Additionally, a variety of tools may be required for initial, interim, and final grip of a device, transfer to the surgical field, and then initial and final placement into the implantation location. Since surgical operating rooms do not necessarily have the same set of surgical instruments, coupled with the natural tendency for surgical users to not want to switch between numerous tools to achieve final placement, there is also a need for a single tool to achieve most or all of these functions.

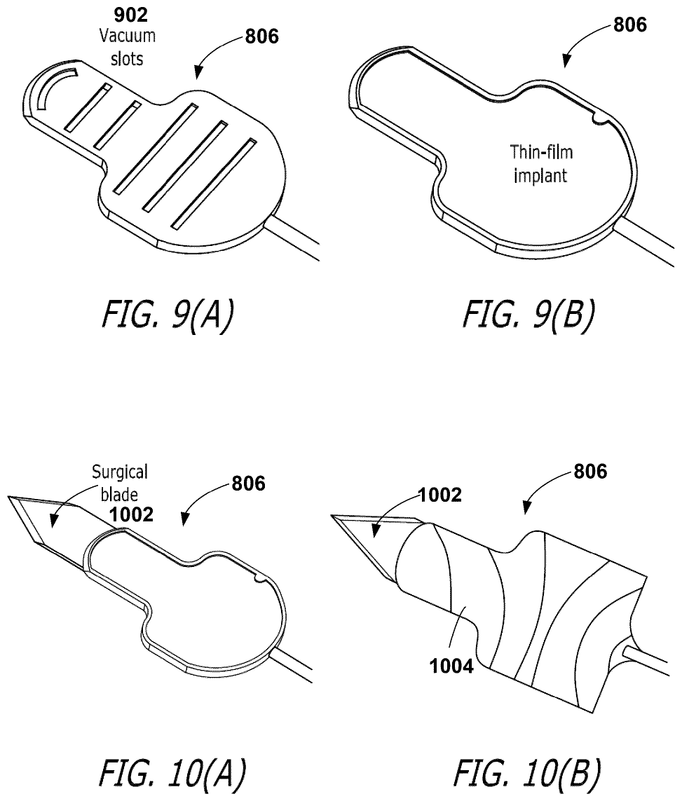

In accordance with aspects of the present disclosure, FIG. 8 shows a delivery device or tool 800 including a handle portion 802 to enable control of a vacuum source and a shaft portion 804 to transmit vacuum from a vacuum source to a tip portion 806 of the delivery tool 800. The handle portion 802 may also include a blade control 810 for controlling a deployable surgical blade as shown in FIGS. 10(A) and 10(B). The tip portion 806 may be customized to match most or all of thin-film implant shapes. FIGS. 9(A) and 9(B) illustrate a close-up view of the tip portion 806 without and with a thin-film implant in position, respectively. In one embodiment, the tip portion 806 may include a plurality of vacuum slots 902, as shown in FIG. 9(A).

In some embodiments, referring back to FIG. 8, the handle portion 802 of the delivery tool 800 may be configured to contain a vacuum source and controls 808 of the vacuum source. The vacuum is a differential in pressure in the operating field and that within the tool tip, such that a thin-film implant (e.g., treatment device 1 of FIG. 6) may be retained on the tip portion 806 of the delivery device 800 throughout the installation process. In some implementations, it does not need to be an absolute vacuum, and even a small partial differential may be sufficient to grip the implant throughout the process. This vacuum may be pulled manually by a user of the delivery device 800 or operated by a switch which can activate an internal pump.

The vacuum may be transmitted from the handle portion 802 to the tip portion 806 of the tool 800 through the shaft portion 804 via the media of air or with a suitable fluid such as water, saline, basic salt solution, viscoelastic, etc. It should be appreciated that the media must be suitable for contact with the tissues of the surgical site such as the eye of a patient.

In one aspect, the vacuum of the delivery device 800 may be created on board via a mechanism such as a syringe and plunger integrated with the handle portion 802. Alternatively, a battery or alternating current (AC) powered vacuum pump may be housed within the handle portion 802 to create and maintain vacuum. In yet another embodiment, a vacuum supply from the hospital facility or operating room may be connected to the handle portion 802 and regulated in either a binary, digital, or analog way with a control mechanism in the handle portion 802 to control the vacuum.

The hollow tool tip portion 806 may be configured to provide a platform on which a thin-film implant is mounted, either in manufacturing or in the operating theater. The vacuum may be engaged before the implant is mounted or afterwards. The pressure differential is transmitted through the hollow tip 806 via open slots (e.g., vacuum slots 902 shown in FIG. 9(A)) on the contact face onto which the thin-film implant is mounted. In a preferred embodiment, no leaks may be present when the vacuum is engaged and the slots are completely covered. Minor leaks may not be an issue if the vacuum source (pump or house vacuum) may overcome the leaks and still provide sufficient grip. Aspiration of air and/or surgical site fluids into the delivery device may be likely if leaks are present. If liquid aspiration risks undermining the vacuum by filling the internal reservoir, the delivery device of the present disclosure may be configured to have a fluid catch container installed or built separately via appropriate vacuum connectors and tubing (F-shaped, T-shaped, Y-shaped, X-shaped vacuum connectors) to allow continuous vacuum.

To release the thin-film implant, a user may reduce or fully release the vacuum via the handle control. Further, a positive pressure may be applied via the handle portion 802 to push or lift the thin-film implant off the tool tip portion 806. The tool may be then removed from the surgical site, leaving the thin-film implant in its final configuration.

As shown in FIGS. 9(A) and 9(B), the implant face of the tip portion 806 of the delivery device may be configured to match the profile of the thin-film implant and support at least a portion of all critical areas of the implant, as required. In some implementations, the thin-film implant may overhang the tip if certain areas of the implant are not critical and/or do not need support. Alternatively, one tool tip design may support a variety of implant sizes if they all fit onto tip without significantly exposing the vacuum slots. The edges of the tip portion 806 may be rounded, beveled, or chamfered at the distal and/or proximal faces to facilitate smooth and easy entry and exit to and from the surgical site. It should be appreciated that the dimensions and shapes of the tip portion 806 may be determined in accordance with that of the thin-film implant. In one aspect, the tip portion 806 of the tool should be thin. For example, the tip portion 806 may be in the range of approximately 0.1 to 5 mm, preferably about 0.5 mm thin with a hollow core of about 0.25 mm in height, and made from with a biocompatible material such as appropriate polymers like polycarbonate, polyethylene, etc., or metals, such as stainless steel or aluminum.

The perforations within the tool may be sized and distributed to ensure that a sufficient grip on the thin-film implant is maintained during a surgical procedure without the handling by the user or tissues at the surgical site causing the implant to be dislodged, wrinkled or damaged. The perforations may be an array of one or more holes between 0.01 to 2 mm in terms of its inner diameter, linear slots in which each side is between 0.1 to 5 mm, or may have other suitable geometry that ensures optimal transmission of the vacuum while maintaining support of the thin-film implant. A preferred embodiment may include one or more 0.25 mm×2 mm rectangular slots. In alternate embodiments, the tip-to-implant face may include a woven mesh or weave of polymeric fibers that effectively transmit the vacuum. Some orifice designs may be included on the tip-to-implant face, such that regions of the thin-film implant may be pulled below the face of the tool tip to protect the edges of the implant from being dislodged by the handling or insertion process.

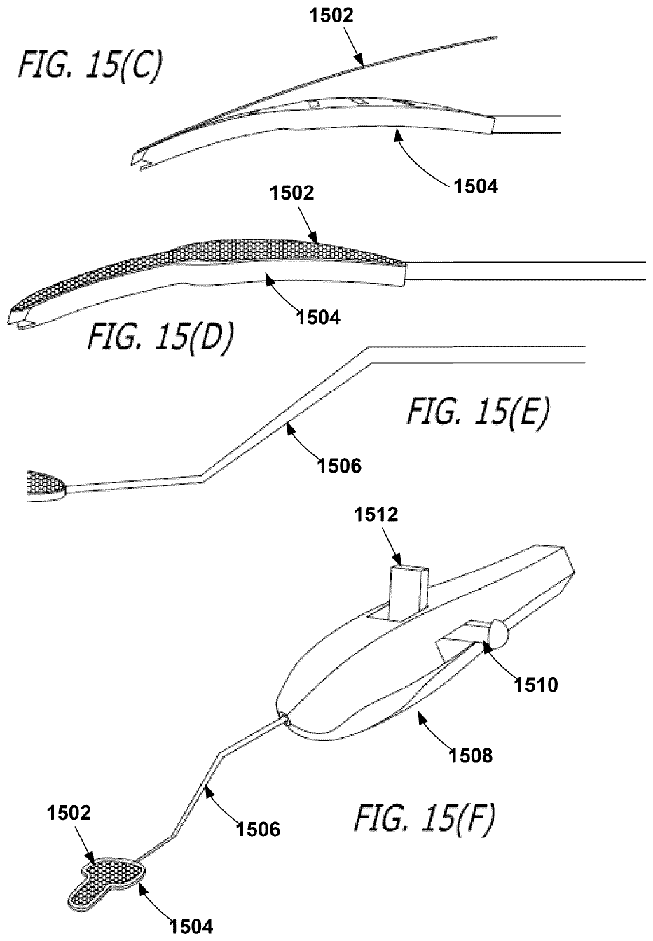

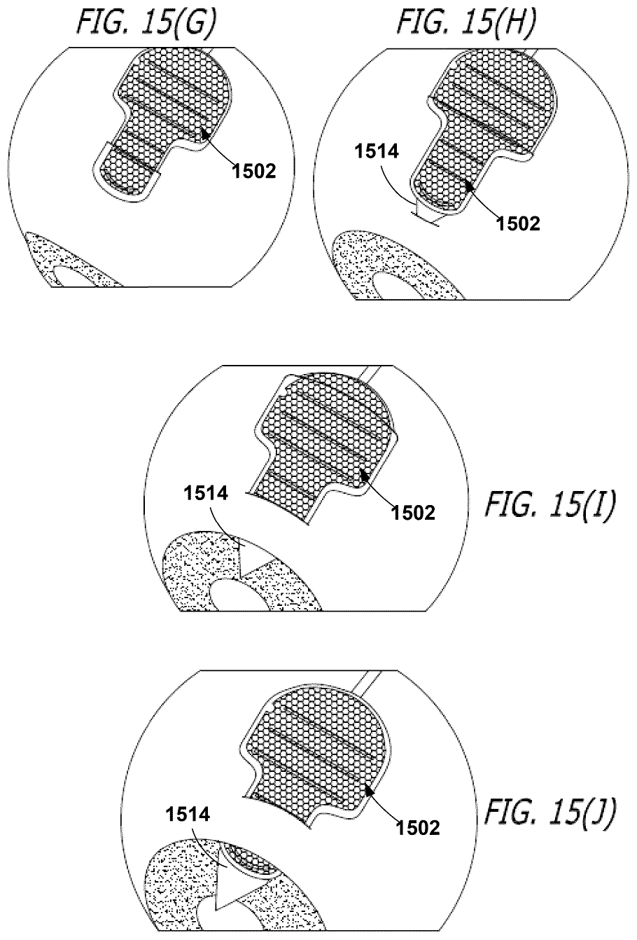

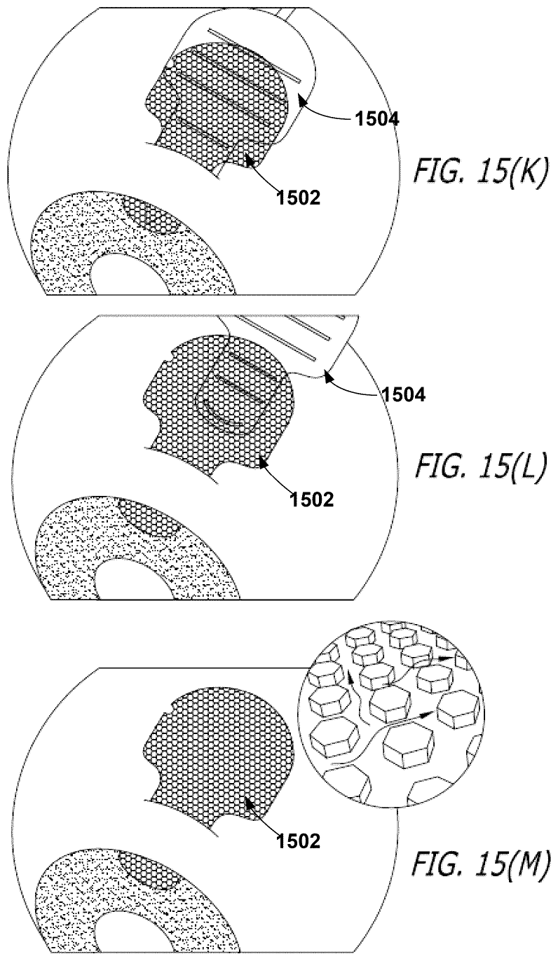

The tool tip face may be planar or non-planar. In one embodiment, a non-planar tip surface such as that shown in FIGS. 15(B), 15(C), and 15(D), may facilitate grip and delivery of at least one thin-film implant into a surgical site. In the context of a glaucoma implant surgery, the surgical site may be curved, such as the globe of the eye, or the thin-film implant may have surface features for which the tip face must accommodate. As a result, the tip face and an array of vacuum perforations may be non-planar to accommodate these surface features and characteristics of the surgical site and the implant.

Another non-planar configuration may include a shallow nest in which the thin-film implant resides while some or all of the perimeter of the tool tip is at or slightly higher than the top most plane of the thin-film implant. As a result, the edges of the thin-film implant may be hidden from the tissue structures which may tend to pull the implant off the tool tip during deployment.

Still referring to FIG. 8, the shaft portion 804 of the tool 800 may be configured to separate the handle portion 802 at a distance from the tip portion 806 to accommodate the need to keep a larger handle further away from the surgical site. In one embodiment, the shaft portion 804 may be a hollow structure between 1-10 cm or more in length with an inner diameter of about 0.1 to 5 mm and a slightly larger outer diameter. In a preferred embodiment, the shaft portion 804 may be 5 cm in length with a 19 gauge shaft size of about 1 mm in terms of its outer diameter×0.7 mm for its inner diameter. The shaft portion 804 may be a hollow round tube or may have other non-circular cross section. Stainless steel hypodermic tubing is a common material used in medical devices for these purposes, but other tubing material such as suitable polymers or metals may be appropriate. As shown in FIG. 8, the shaft portion 804 may include multiple bends (e.g., two bends 812, 814) along its length to offset the plane of the tool tip 806 from the longitudinal axis of the handle portion 802, thereby allowing clearance for the handle 802 and/or user's fingers from contacting the anatomical structures around the surgical sites such as the brow ridge or nose in the case of an ocular surgery. The shaft portion 804 may be made of an articulating structure or clastic material so as to allow the user to adjust the degree of offset from the handle shaft to the tip. This may be achieved via an internal pull-wire within the shaft portion 804 that is anchored at one location near the tool tip 806 or a selected distal location of the desired deflection point on the shaft 804 and a second location within the handle portion 802 such as at a lever or slider to allow deflection adjustment.

In another embodiment as shown in FIG. 10(A), a deployable surgical blade 1002 may be integrated into the tool of FIG. 9(A), in accordance with aspects of the present disclosure. FIG. 10(B) shows an example implementation of the prototype of FIG. 10(A). Specifically, the tool tip 806 of the present disclosure may incorporate a surgical blade 1002 of a geometry that is selected to facilitate placement of the implant within the surgical site. Such a blade 1002 may be configured to facilitate the placement of the narrow tab like section of the thin-film implant within a scleral tunnel. The surgical blade 1002 may be affixed to the handle portion 802 via an actuation member that is secured to an actuation lever within the handle portion, according to one embodiment. The actuation member may reside within the tool shaft or alongside it. A user may deploy the blade 1002 from a sheath 1004 or the bottom face of the tool tip 806, such that it is exposed and allows the user to make the appropriate surgical cut(s), and then retract it for subsequent positioning of the thin-film implant. In a preferred embodiment, the blade 1002 may be thin (e.g., 0.1-1.0 mm thick) and no wider than the tool tip 806. The degree to which is deployed may be determined based upon the necessary depth of the required cut, ranging between 1-10 mm in length.

In accordance with other aspects of the present disclosure, the incorporation of braces within the tool tip 806 may enable it to resist the mechanical forces associated with surgical handling and the deployment of vacuum. As shown in an exploded view of section 1102 of FIG. 11, one or more internal braces may span the top face to the bottom face between slots to make the faces remain apart and planar from one another. Otherwise the deflection or collapse of the vacuum face may inhibit the transmission of vacuum or cause mechanical failure of the tool tip 806. The internal braces may include ribs, posts or similar structures embedded within the plate, wall or a fibrous mesh that is transmitting the vacuum and supporting the vacuum face of the tip portion 806.

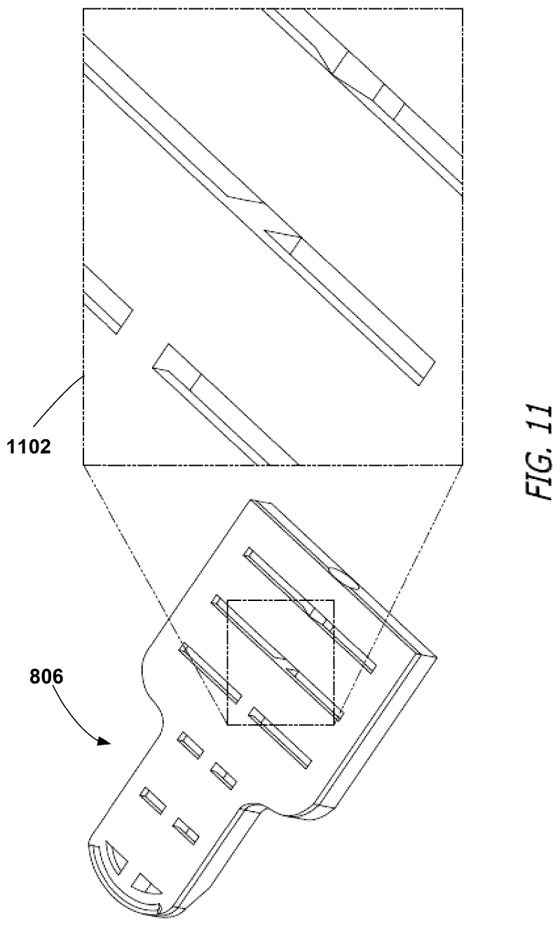

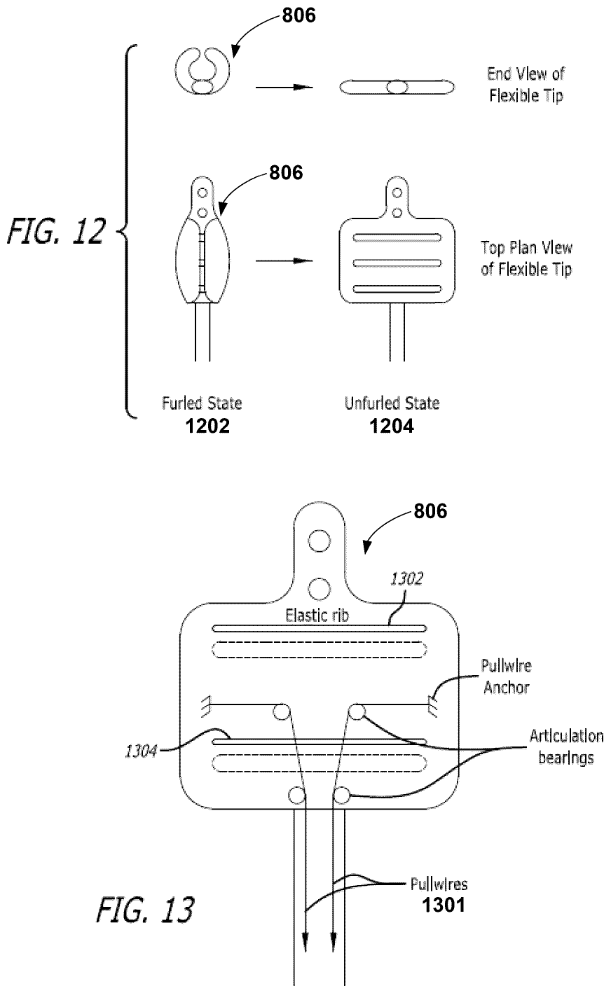

Referring now to FIG. 12, a minimally invasive tool tip 806 may be constructed from clastic or articulating parts or materials in accordance with aspects of the present disclosure, such that the tip portion 806 of the tool may have 2 main configurations. One configuration may generally refer to a furled or collapsed configuration 1202 in which the vacuum tool tip 806 is rolled with the flexible thin-film implant adhered to its face. As for the second configuration 1204, the tip portion 806 may be unfurled in a geometry that is most suitable for the final implant placement. For example, the tool may be delivered in a minimally invasive manner in configuration 1202 through a prepared surgical site that is significantly smaller than the final configuration 1204, which offers advantages of minimal tissue disruption, bleeding, time to prepare a larger surgical site, and patient pain or discomfort.

To enable the furled (1202) or unfurled (1204) configuration of the minimally invasive tool tip 806, a mechanism to induce the curling of the tool face may be required. For example, referring to FIG. 13, one or more pull wires 1301 may be secured at one end to appropriate locations on the tool face and then run alongside or inside the tool shaft to a lever or button within the handle. As shown in FIG. 13, a pair of pull wires 1301 may be arranged, such that tension placed upon them via a handle mechanism can cause the selective deformation of the tool face to achieve a smaller profile for the placement of the thin film implant in a minimally invasive manner. Lines 1302, 1304 depict one or more elastic ribs configured to enable the unfurled configuration 1204 shown in FIG. 12, while being capable of easily bending elastically to the furled configuration 1202 when pull-wire tension is applied. The lever at the handle may allow the user to selectively curl and straighten the tool tip 806 accordingly. That is, in certain cases, when the tool tip 806 is in a unfurled configuration 1204, it may be difficult to push on a pull wire 1301 to have the configuration become flat. Therefore, integration of the clastic struts 1302, 1304 such as those shown in FIG. 13 may facilitate the unfurled configuration 1204. One embodiment is the integration of selective wire struts made from Nitinol within the tool tip. When tension is applied to the pull wires 1301, these Nitinol struts easily bend elastically to a very tight configuration and allow the wings of the tool tip 806 to curl. When the tension is released in the pull-wires 1301, the Nitinol struts straighten out and restore the tool tip 806 to its flat and expanded profile.

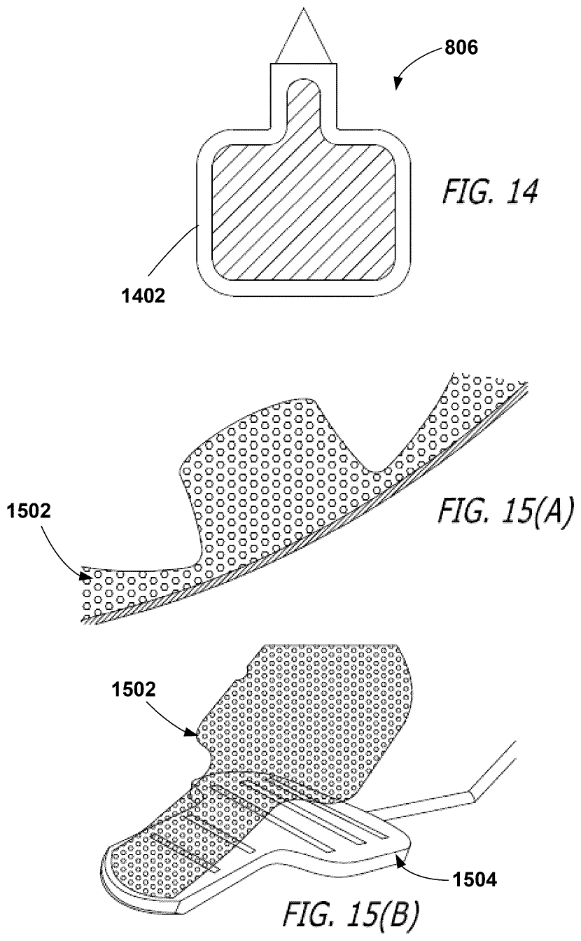

FIG. 14 shows a sheath configuration of the tool tip 806 which includes an additional support plate or sheath component 1402 configured to shield the thin-film implant during delivery process, in accordance with aspects of the present disclosure. The additional support plate 1402 secures the thin-film implant and aids in the withdrawal of the insertion tool from the scleral tunnel, thus fully enclosing a flat system. The thin-film implant may be protected during insertion into the scleral tunnel, and the top and bottom pieces withdrawal independently such that the thin-film implant may stay fixed in the tunnel when the inserter is removed. In one embodiment, the top and bottom pieces may both utilize vacuum independently. The thin-film implant resides between the sheath and the tool tip platform, so as to additionally shield the implant during the delivery process. In some implementations, either the sheath 1402 or tool tip 806 may be retracted independently once the vacuum is released. This may enable the thin-film implant to reside more stably in the surgical site upon retraction of the delivery tool.

Manufacturing of the tool tip 806 of the present disclosure may be accomplished by micro injection molding, micro 3D printing, fine wire electrical discharge machining (EDM), or micro computerized numerical control (CNC) machining among others. In one embodiment, a tool tip of the present disclosure may be manufactured via micro 3D printing. The tool tip 806 may include multiple parts to achieve the final assembly configuration that adequately transmits a vacuum, while having the requisite strength to withstand the handling and vacuum forces. The thin-film implant may be preloaded in manufacturing onto the tool tip 806 and provided to users in a sterile configuration, or a user may install the thin-film implant separately onto the tool.

FIGS. 15(A), 15(B), 15(C), 15(D), 15(E), 15(F), 15(G), 15(H), 15(I), 15(J), 15(K), 15(L), and 15(M) are screenshots showing how the delivery tool of the present disclosure may be used to handle and deliver at least one thin-film implant into a surgical site that is open or minimally invasive (e.g., an ocular surgical site). FIG. 15(A) shows an example thin-film implant 1502 (e.g., treatment device 1 of FIG. 6) of the present disclosure. FIGS. 15(B), 15(C), and 15(D) illustrate the thin-film implant 1502 being installed onto a delivery tip 1504 of the delivery tool of the present disclosure in incremental stages. FIG. 15(E) shows a side view of the delivery tool shaft portion 1506. FIG. 15(F) is a perspective view of the delivery tool, which has a handle portion 1508 and multiple actual levers 1510, 1512 to deploy the vacuum and an insertion blade. FIG. 15(G) shows a system (the delivery tool and the thin-film implant 1502) being deployed at a surgical site such as an eye of a patient, where a scleral pocket has been previously prepared. FIG. 15(H) shows the tip of the surgical blade 1514 being deployed into the scleral tissue to create a scleral tunnel for placement of the narrow portion of the thin-film implant 1502. FIG. 15(I) shows the blade tip 1514 completing the tunnel into the anterior chamber where the tip of the thin-film implant 1502 will communicate. FIG. 15(J) shows the insertion of the delivery tool with the thin-film implant into the ocular anterior chamber, while FIG. 15(K) shows the retraction of the surgical blade 1514. At this time the vacuum may be released or diminished or replaced with positive pressure to facilitate detachment of the thin-film implant 1502 from the tool tip 1504. FIG. 15(L) shows the partial retraction of the delivery tool, while the thin-film implant 1502 remains in the target surgical site, where the thin-film implant tip extends from the anterior chamber, through the scleral tunnel and into the subconjunctival pocket. FIG. 15(M) shows the final position of the thin-film implant 1502 where the delivery tool has been removed from the surgical site and the inset depicts aqueous humor flowing along the surface of the thin-film implant 1502 from the anterior chamber to a scleral bleb to lower the intraocular pressure.

In accordance with additional aspects, the delivery tool of the present disclosure may include a number of features such as monitoring and communicating the state of the vacuum. If a leak occurs or the thin-film implant is disrupted, for example, the vacuum of the delivery tool may be partially or completely released without the user knowing it by the appearance of the tool tip. In some embodiments, the handle portion of the delivery tool may be configured to include a monitor such as a pressure transducer in the flow path of the vacuum to detect the level of vacuum. Another feature such as an electrical circuit or analog gauge may evaluate the electrical value of the transducer to display via a light, a light-emitting diode (LED), or LED-display the vacuum status and indicate an analog or digital value via numbers or colors or other visual or audio signals to show if the vacuum is appropriate for security of the thin-film implant. For example, in response to detecting that the vacuum level is appropriate, the display may be configured to indicate a green light or negative pressure value. If the vacuum level is not in place or sufficient, the display may indicate a red light, a “0” or positive value, for example. The dimensions, shape, and location of the display on the delivery tool may be determined in accordance with certain selected characteristics. For example, if sufficiently small, the display may be implemented and installed at the handle portion, the shaft portion, and/or tool tip portion to communicate to the user, particularly when the user's vision is fixated at the tool tip through a surgical microscope rather than the device handle. The user may re-establish vacuum as required before committing to the installation procedure.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the application (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the application and does not pose a limitation on the scope of the application otherwise claimed. No language in the specification should be construed as indicating any non-claimed element essential to the practice of the application.

Groupings of alternative elements or embodiments of the application disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this application are described herein, including the best mode known to the inventors for carrying out the application. Of course, variations on these described embodiments will become apparent to those of ordinary skill in the art upon reading the foregoing description. The inventor expects skilled artisans to employ such variations as appropriate, and the inventors intend for the application to be practiced otherwise than specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the application so claimed are inherently or expressly described and enabled herein.

In closing, it is to be understood that the embodiments of the application disclosed herein are illustrative of the principles of the present application. Other modifications that may be employed are within the scope of the application. Thus, by way of example, but not of limitation, alternative configurations of the present application may be utilized in accordance with the teachings herein. Accordingly, the present application is not limited to that precisely as shown and described.

1. A device, comprising:
a tip portion configured to secure and protect a thin-film implant device during handling and installation into a surgical site;
a handle portion configured to control a vacuum source; and
a shaft portion configured to connect the handle portion and the tip portion and control and deliver vacuum from the vacuum source to the tip portion.

2. The device of claim 1, wherein the vacuum source is housed in the handle portion of the device.

3. The device of claim 1, wherein the vacuum source is external to the device.

4. The device of claim 1, wherein the tip portion, the shaft portion, and the handle portion are located substantially in a same plane and form a straight configuration.

5. The device of claim 1, wherein the tip portion, the shaft portion, and the handle portion are located in different planes with the shaft portion including multiple bends along its length to offset a first plane of the tip portion from a longitudinal axis of a second plane of the handle portion.

6. The device of claim 1, wherein the vacuum source includes a power supply, wherein the power supply includes at least one of a syringe pump, a battery, or an alternating current source.

7. The device of claim 1, wherein the tip portion is configured to include a blade tip implemented on a distal end to prepare surgical installation of the thin-film implant device.

8. The device of claim 7, wherein the blade tip is deployable and connected to the handle portion via an actuation member that is secured to an actuation lever within the handle portion.

9. The device of claim 1, further comprising at least one pressure transducer positioned in a flow path of vacuum for detecting a level of vacuum of the vacuum source.

10. The device of claim 9, further comprising a display configured to display the level of vacuum detected by the at least one pressure transducer.

11. The device of claim 10, wherein the display is implemented and installed at one of the tip portion, the shaft portion, or the handle portion.

12. The device of claim 10, wherein the display is configured to display the level of vacuum via one or more analog signals or digital signals.

13. The device of claim 12, wherein the display includes a light-emitting diode (LED) display.

14. The device of claim 12, wherein the one or more analog signals or digital signals comprise visual or audio signals configured to indicate the level of vacuum detected by the at least one pressure transducer.

15. The device of claim 1, wherein the tip portion includes a plurality of vacuum slots.

16. The device of claim 1, wherein the tip portion has dimensions and a shape configured to accommodate different sizes of the thin-film implant device.

17. The device of claim 1, wherein edges of the tip portion at distal and proximal faces are rounded, beveled or chamfered to facilitate a smooth and easy entry and exit to and from the surgical site.

18. The device of claim 1, wherein the tip portion is made of biocompatible materials.

19. The device of claim 1, wherein the tip portion includes an array of perforations configured to regulate vacuum delivery while maintaining a support of the thin-film implant device.

20. The device of claim 1, wherein the tip portion includes a tip-to-implant face configured to be non-planar.

21. The device of claim 20, wherein the tip-to-implant face includes a woven mesh or weave of polymeric fibers.

22. The device of claim 20, wherein the tip-to-implant face includes a shallow nest in which the thin-film implant device resides while at least a portion of a perimeter of the tip portion is higher than a top most plane of the thin-film implant device.

23. The device of claim 1, wherein the tip portion is configurable between a furled state and a unfurled state, wherein the tip portion is rolled with the thin-film implant device adhered to a top surface of the tip portion in the furled state, and the tip portion is in the unfurled state for a placement of the thin-film implant device into a surgical site.

24. The device of claim 23, wherein the tip portion includes one or more wires extending within the shaft portion and connected with at least one component within the handle portion, each wire having a first end secured on a selected location on the top surface of the tip portion and a second end attached to the at least one component.

25. The device of claim 24, wherein the at least one component is configured to control tension of the one or more wires.

26. The device of claim 24, wherein the at least one component is a lever or a button implemented on the handle portion.

27. The device of claim 23, wherein the tip portion is made of elastic or articulating parts of materials to enable the furled and unfurled states of the tip portion.

28. The device of claim 23, wherein the tip portion includes one or more elastic struts to enable the unfurled state.
